(19) United States
(12) Patent Application Publication
(10) Pub. No.: US 2024/0260826 A1
(43) Pub. Date: Aug. 8, 2024

(54) UV LUMINAIRE DEVICE FOR DENTISTRY APPLICATION

(71) Applicant: DENTOZONE CO., LTD., Seoul (KR)

(72) Inventor: Cha Hyoung KOO, Seoul (KR)

(21) Appl. No.: 18/015,433

(22) PCT Filed: Dec. 17, 2021
(86) PCT No.: PCT/KR2021/019258

§ 371 Date: Jan. 10, 2023

(30) Foreign Application Priority Data
Nov. 29, 2021 (KR) 10-2021-0167256

(51) Int. Cl. A61B 1/24; A61B 1/06

(52) U.S. Cl. CPC A61B 1/24; A61B 1/06

(57) ABSTRACT

A UV luminaire device for a dentistry application according to an embodiment includes a housing provided with an installation space allowing a luminaire module to be embedded therein, a luminaire part installed on a front surface of the housing and configured to emit light radiated from the luminaire module to the outside, and a mounting means installed to protrude from a lower portion of the housing and capable of being mounted on eyeglasses.

### CROSS REFERENCE TO RELATED APPLICATIONS AND CLAIM OF PRIORITY

This application claims benefit under 35 U.S.C. 119, 120, 121, or 365 (c), and is a National Stage entry from International Application No. PCT/KR2021/019258 filed on Dec. 17, 2021, which claims priority to the benefit of Korean Patent Application No. 10-2021-0167256 filed in the Korean Intellectual Property Office on Nov. 29, 2021, the entire contents of which are incorporated herein by reference.

### BACKGROUND

### 1. Technical Field

The present disclosure relates to a UV luminaire device for a dentistry application and, more specifically, to a UV luminaire device for a dentistry application for irradiating the inside of an oral cavity of a patient with UV light by being installed on glasses used by a dentist.

### 2. Background Art

Dental examination and treatment are generally performed in the patient's oral cavity, so a luminaire device for illuminating the inside of an oral cavity of a patient has been installed in a dental unit chair.

Luminaire devices used for dental examination and treatment need to generate no shadows to facilitate the examination and treatment, and such medical luminaire devices are referred to as astral lamps.

Conventional astral lamps are made in a structure to prevent an occurrence of shadows by arranging several light bulbs on the light body attached to an upper side of a dental unit chair, wherein light emitted from each light bulb illuminates an affected area in different directions so that a shadow caused by the light from one light bulb is offset by other light bulbs.

However, in a conventional dental luminaire device such as the astral lamp, when different luminous environments are required for treatment such as microvascular treatment, periodontal treatment, implant treatment, and the like, there are problems in that it is not easy to build a luminous environment suitable for the treatment purpose. Such problems are supplemented through Patent Document 1 (KR 10-1926500 B1) and are as follows to describe in detail with reference to Patent Document 1.

Patent Document 1 relates to a glasses-mounted luminaire device, and according to this, the device includes a housing part in which the luminaire device is built, a luminaire part provided on the front surface of the housing part to radiate light generated by the luminaire device, a mounting part provided at a lower portion of the housing part and mounted on eyeglasses, and a coupling clip provided between the housing part to connect the first housing part and the second housing part.

However, the above-described Patent Document 1 is inconvenient to adjust an angle of the luminaire part, and in particular, when the angle of the luminaire part is adjusted for a long time, abrasion or damage is used to occur due to long-time use, causing a problem in that the luminaire part descends.

### SUMMARY

Accordingly, the present disclosure has been made keeping in mind the above problems occurring in the related art, and an objective of the present disclosure is to improve the coupling structure between a luminaire part and a housing in order to improve and supplement Korean Patent Registration No. 10-1926500, and to provide a UV luminaire device for a dentistry application for easily adjusting the angle of the luminaire part.

In order to accomplish the above objective,
there may be provided a UV luminaire device for a dentistry application according to an embodiment of the present disclosure, the device including: a housing provided with an installation space allowing a luminaire module to be embedded therein;
a luminaire part installed on a front surface of the housing and configured to emit light radiated from the luminaire module to the outside; and
a mounting means installed to protrude from a lower portion of the housing and capable of being mounted on eyeglasses,
wherein the housing includes: a fastening part provided with a “c” shape on a front side and provided with a fastening space allowing the luminaire part to be fastened therein;
fastening holes provided on opposite sides of the fastening part and allowing a fastening means to be inserted therein; and
a first through hole provided at the front surface of the housing, thereby allowing a wire to be connected between the housing and the luminaire part, and
the luminaire part includes: a connection part provided with a structure capable of being inserted into the fastening space by protruding downward on one surface thereof and including a fixing hole provided at the inside thereof to allow the fastening means to be inserted therethrough;
insertion grooves provided on opposite outsides of the fixing hole, respectively;
O-rings inserted into the insertion grooves, respectively, and made of a material having flexibility and elasticity; and
a second through hole provided at a protruding surface of the connection part to face the first through hole, thereby allowing the wire to be inserted therethrough so that a battery built in the housing is electrically connected to the lighting module,
wherein a thickness (d2) of the O-ring is provided to be relatively thicker than the thickness (d1) of the insertion groove, and when installed in the insertion groove, the O-ring is provided to protrude from the connection part, and the thickness (d2-d1) that the O-ring protrudes from the connection part is 0.1 mm to 0.9 mm.

In addition, in the dental UV luminaire device according to the embodiment of the present disclosure, the installation space may be provided to have the same size as the luminaire module, and the luminaire module may be installed in the installation space in a fitting manner.

In addition, in the dental UV luminaire device according to the embodiment of the present disclosure, the luminaire part may include: a reflector installed therein and configured to emit the light radiated from the luminaire module to the outside;
a fixing member provided on one surface of the reflector, provided with an installation space allowing the luminaire module to be installed therein, and configured to fix the luminaire module; and
a radiation hole provided between the reflector and a fixing member and provided to allow the light radiated from the luminaire module to be radiated in one direction.

In addition, in the dental UV luminaire device according to the embodiment of the present disclosure, the luminaire module may be installed in the installation space of the fixing member, but an LED installed in the luminaire module is installed to face the radiation hole, thereby allowing the light radiated by the LED to be radiated to the reflector through the radiation hole.

In addition, in the dental UV luminaire device according to the embodiment of the present disclosure, the luminaire module may generate ultraviolet rays (UV) to radiate to the outside, and
a wavelength of ultraviolet rays may be generated within a range of 380 to 390 nm.

In addition, in the dental UV luminaire device according to the embodiment of the present disclosure, the housing ma be provided by being divided into a first housing and a second housing, and the first housing and the second housing may be coupled to each other through a coupling means.

In addition, in the dental UV luminaire device according to the embodiment of the present disclosure, the mounting means may be configured by selecting any one of a clip, a magnet, and tongs.

In addition, in the dental UV luminaire device according to the embodiment of the present disclosure, the fastening means may be configured by selecting any one of a bolt, a screw, and a hinge structure.

In addition, in the dental UV luminaire device according to the embodiment of the present disclosure, the thickness (d2-d1) that the O-ring protrudes from the connection part may be 0.4 mm to 0.6 mm.

In addition, in the dental UV luminaire device according to the embodiment of the present disclosure, the thickness (d2-d1) that the O-ring protrudes from the connection part may be 0.5 mm.

As described above, according to one embodiment of the present disclosure, when a luminaire part is rotated with respect to the housing, as is made in a structure in which an O-ring of elastic material is installed, a connection part is easily rotated, and a certain fixing force is provided by the frictional force generated from the elastic material.

In addition, according to the embodiment of the present disclosure, a fixing member having an installation space in which a luminaire module can be installed is provided on one side of the reflector, and the size of the fixing member is the same as that of the luminaire module, thereby allowing the luminaire module to be prevented from being deviated to the outside or being subjected to minute movement and having an effect of preventing the direction in which light radiates from being distorted or damaging the LED.

### BRIEF DESCRIPTION OF THE DRAWINGS

FIGS. 1 and 2 are perspective views showing a UV luminaire device for a dentistry application according to an embodiment of the present disclosure.

FIGS. 3 and 4 are sectional views showing an installation state of a luminaire module of the dental UV luminaire device according to the embodiment of the present disclosure.

FIG. 5 is a perspective view showing a luminaire part of the dental UV luminaire device according to the embodiment of the present disclosure.

FIGS. 6 and 7 are sectional views showing a fastening state of a housing and a luminaire part of the UV dental luminaire device according to the embodiment of the present disclosure.

### DETAILED DESCRIPTION

There may be provided a UV luminaire device for a dentistry application according to an embodiment of the present disclosure, the device including: a housing provided with an installation space allowing a luminaire module to be embedded therein;
a luminaire part installed on a front surface of the housing and configured to emit light radiated from the luminaire module to the outside; and
a mounting means installed to protrude from a lower portion of the housing and capable of being mounted on eyeglasses,
wherein the housing includes: a fastening part provided with a “c” shape on a front side and provided with a fastening space allowing the luminaire part to be fastened therein;
fastening holes provided on opposite sides of the fastening part and allowing a fastening means to be inserted therein; and
a first through hole provided at the front surface of the housing, thereby allowing a wire to be connected between the housing and the luminaire part, and
the luminaire part includes: a connection part provided with a structure capable of being inserted into the fastening space by protruding downward on one surface thereof and including a fixing hole provided at the inside thereof to allow the fastening means to be inserted therethrough;
insertion grooves provided on opposite outsides of the fixing hole, respectively;
O-rings inserted into the insertion grooves, respectively, and made of a material having flexibility and elasticity; and
a second through hole provided at a protruding surface of the connection part to face the first through hole, thereby allowing the wire to be inserted therethrough so that a battery built in the housing is electrically connected to the lighting module,
wherein a thickness (d2) of the O-ring is provided to be relatively thicker than the thickness (d1) of the insertion groove, and when installed in the insertion groove, the O-ring is provided to protrude from the connection part, and the thickness (d2-d1) that the O-ring protrudes from the connection part is 0.1 mm to 0.9 mm.

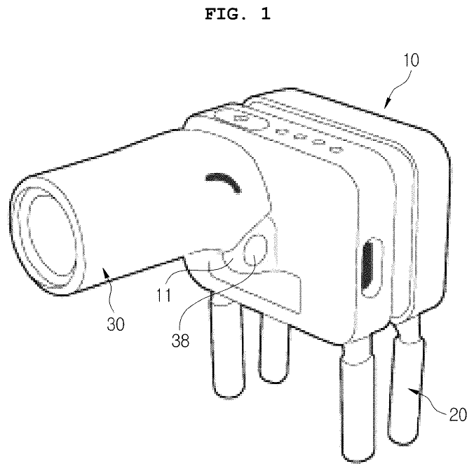

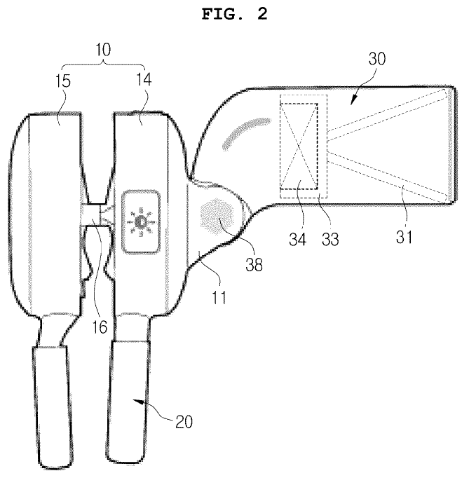

As shown in FIGS. 1 and 2, a dental UV luminaire device according to an embodiment of the present disclosure may include a housing 10, a luminaire part 30, and a mounting means 20.

The housing 10 is provided with an inner space in which the luminaire module 34 may be embedded and may be an outer side cover having a hexahedral shape with rounded corners, but is not limited thereto and may be provided as an outer side cover having various polygonal or circular shapes.

In addition, the housing 10 may be provided by being divided into a first housing 14 and a second housing 15, and a coupling means 16 configured to couple a housing gap between the first housing 14 and the second housing 15 may be provided. At this time, as the coupling means 16, any one of a clip, a bolt, a screw, and a locking device may be selected and coupled to each other.

The mounting means 20 may be provided to protrude from a lower portion of the housing 10, may be provided with, but not limited to, a “ [symbol] ” shape so as to be worn on glasses, and may be provided with various means such as clips, magnets, tongs, and the like.

The luminaire part 30 may be installed on the front of the housing 10 and may be provided with a cylindrical shape, and a luminaire module 34 may be installed inside to emit light radiated from the luminaire module 34 to the outside.

In addition, the luminaire part 30 has a connection part 35 provided at the bottom on one side (a position close to the housing in the present disclosure) and may be fastened through the fastening means 38 after the connection part 35 is fastened in a fitting manner into the fastening part 11 provided on the front surface of the housing 10. At this time, the luminaire part 30 may be rotated up and down from the housing 10 by the fastening means 38 serving as an axis.

Such a luminaire part 30 may be provided with a structure allowing a reflector 31 and a fixing member 33 to be embedded therein and a luminaire module 34 to be installed in the fixing member 33.

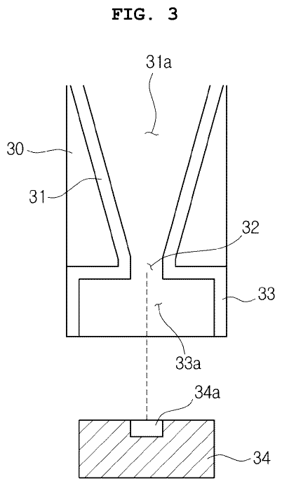

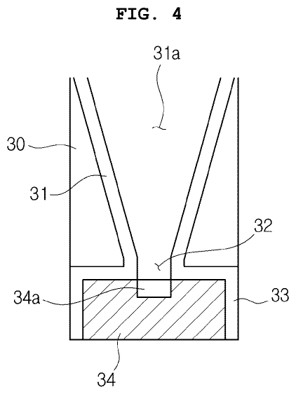

Specifically, as shown in FIGS. 3 and 4, the luminaire part may be provided with a reflector installed therein, the reflector having an aperture on one side relatively larger than an aperture on an opposite side (a structure in which the lower portion is formed relatively smaller than the upper portion based on FIGS. 3 and 4) and being allowed to emit light provided from the luminaire module 34 to the outside, and may have a fixing member 33 installed on one side thereof (a lower portion based on FIGS. 3 and 4).

The reflector 31 may be configured such that a reflection space 31a is provided, thereby allowing light radiated through the radiation hole 32 to be emitted to the outside.

The fixing member 33 may be provided at an entrance of the reflector 31, provided with the same size as the luminaire module 34, and provided with an installation space allowing the luminaire module 34 to be built-in. The installation space may be provided to have the same size as the luminaire module 34, thereby allowing the luminaire module 34 to be rigidly fixed by being fastened in a fitting manner.

A radiation hole 32 may be provided between the reflector 31 and the fixing member 33 so that the light emitted from the luminaire module 34 is radiated in one direction (outside), and an LED 34a generating light in the luminaire module 34 may be installed to be aligned with the direction of the radiation hole 32.

Specifically, the luminaire module 34 may include an LED 34a, and the luminaire module may be configured such that the light generated by the LED 34a is emitted through the radiation hole 32 and a reflector 31 to be emitted to the outside since the LED 34a is installed at the same position as the radiation hole 32.

Here, the LED 34a may generate and radiate ultraviolet rays (UV) to the outside, and the wavelength of the ultraviolet rays may be generated within a range of 375 to 410 nm, and when further limited, within a range of 380 to 390 nm.

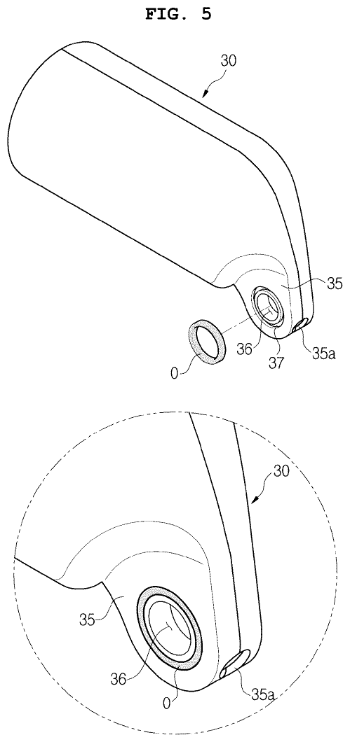

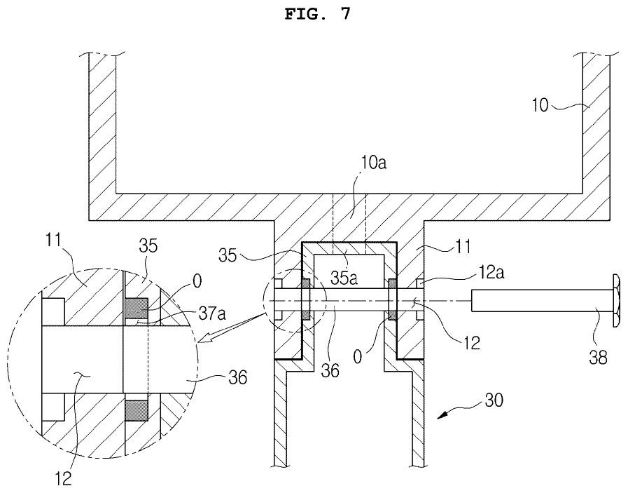

In addition, the coupling relationship between the housing 10 and the luminaire part 30 may be explained as shown in FIGS. 5 to 7.

The housing 10 may include: a first through hole 10a provided on a front surface and provided with a hole shape to allow a battery (not shown) provided inside the housing 10 and the luminaire module 34 to be interconnected by wiring; a fastening part 11 provided with a “c” shape at a lower portion and provided with a fastening space 13 allowing the mounting means 20 to be fastened therein; and fastening holes 12 provided on opposite sides of the fastening part 11 and allowing a fastening means 38 to be inserted thereinto and fastened thereto.

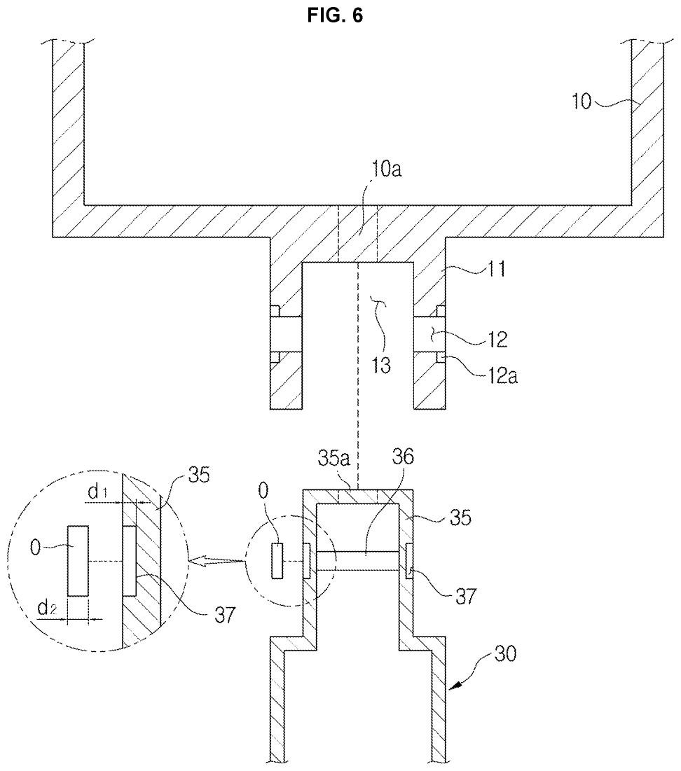

The fastening holes 12 are each a hole provided to allow the fastening means 38 to be penetrated through, and head grooves 12a, in which heads of the fastening means 38 may be respectively installed, may be provided on opposite sides of the fastening part 11 near the fastening holes 12 (more specifically, surfaces opposite to the fastening space 13 as shown in FIG. 6), respectively.

The fastening means 38 may be configured by selecting any one of a hinge structure, a bolt, and a screw and may be provided with the hinge structure in the present disclosure.

The fastening means 38 may serve as an axis after being inserted into the fastening hole 12 to allow the luminaire part 30 to be rotated from the housing 10.

The luminaire part 30 is inserted into the fastening space 13, and the fixing hole 36 provided with the luminaire part 30 and the fastening hole 12 provided with the fastening part 11 are provided to be aligned the same as each other and may be structured to be fastened by allowing the fastening means 38 to be inserted therethrough.

Here, the luminaire part 30 may further include: a connection part 35 structured to be inserted into the fastening space 13 with one side protruding downward and provided with a fixing hole 36 therein; insertion grooves 37 provided on opposite outsides of the fixing hole 36, respectively, and provided with a space into which an O-ring O may be inserted; and a second through hole 35a provided at a lower portion of the connection part 35 to allow a wire between the luminaire part 30 and the housing 10 to be connected.

The second through hole 35a may be configured to allow the battery and the luminaire module 34 to be connected through a wire so as to allow the battery (not shown) built in the housing 10 to supply power to the luminaire module 34 built in the luminaire part 30.

The insertion grooves 37 may each be provided with a groove shape near the fixing hole 36 so as to be provided with a space into which an O-ring O made of flexible and elastic material may be inserted.

The O-ring O may be installed by being inserted into the insertion groove 37 and may be installed to be spaced apart from the fixing hole 36. That is, the O-ring O may be configured such that when the O-ring O is installed in the insertion groove 37, the O-ring O comes into close contact with the inner wall 37a of the insertion groove. Furthermore, the inner wall 37a of the insertion groove may be configured to be aligned with the inner wall of the fixing hole 36.

In addition, the O-ring O is provided so that the depth of the groove provided at the insertion groove 37 is relatively smaller than the thickness of the O-ring O, so when installed in the insertion groove 37, the O-ring O may be provided with a structure in which a portion thereof protrudes to a certain portion from the connection part 35.

Specifically, the thickness d2 of the O-ring O is provided relatively larger than the thickness d1 of the insertion groove 37, and when inserted into and installed at the insertion groove 37, the O-ring O may be configured such that a portion thereof protrudes compared with a portion of the connection part 35. At this time, the thickness d2-d1 that the O-ring O protrudes from the connection part 35 may be made of 0.5 mm, for example, when the thickness d2-d1 exceeds 0.5 mm, the frictional force is provided more than necessary, so the luminaire part 30 may be made of a structure that is difficult to rotate smoothly, and when the thickness d2-d1 is provided to be less than 0.5 mm, the frictional force is not provided, so the effect of installing the O-ring O may be insufficient.

Here, the thickness d2-d1 at which the O-ring O protrudes from the connection part 35 may be provided differently in proportion to the size of a luminaire device 1, but a typical luminaire device 1 is 0.1 mm to 0.9 mm, and more specifically, 0.4 mm to 0.6 mm.

After this O-ring O is installed in the insertion groove 37 to protrude from the connection part 35 by a certain portion (with 0.5 mm as a reference), when the connection part 35 is installed in the fastening part 11, the ring O is installed in a pressed state by the fastening part 11. Accordingly, frictional force may be generated using the elastic material of the O-ring O, so that fine angle adjustment is possible, and the O-ring O has the effect of providing a fixing force to fix to the housing through frictional force no less than a certain degree.

The present disclosure may be used in the medical field and, more specifically, may be used by dentists in dental hospitals.

1: A ultraviolet (UV) luminaire device for a dentistry application, the device comprising:
a housing provided with an installation space allowing a luminaire module to be embedded therein;
a luminaire part installed on a front surface of the housing and configured to emit light radiated from the luminaire module to the outside; and
a mounting means installed to protrude from a lower portion of the housing and capable of being mounted on eyeglasses,
wherein the housing comprises:
a fastening part provided with a “c” shape on a front side and provided with a fastening space allowing the luminaire part to be fastened therein;
fastening holes provided on opposite sides of the fastening part and allowing a fastening means to be inserted therein; and
a first through hole provided at the front surface of the housing, thereby allowing a wire to be connected between the housing and the luminaire part,
wherein the luminaire part comprises:
a connection part provided with a structure capable of being inserted into the fastening space by protruding downward on one surface thereof and including a fixing hole provided at the inside thereof to allow the fastening means to be inserted therethrough;
insertion grooves provided on opposite outsides of the fixing hole, respectively;
O-rings inserted into the insertion grooves, respectively, and made of a material having flexibility and elasticity; and
a second through hole provided at a protruding surface of the connection part to face the first through hole, thereby allowing the wire to be inserted therethrough so that a battery built in the housing is electrically connected to the lighting module,
wherein a thickness (d2) of the O-ring is provided to be relatively thicker than the thickness (d1) of the insertion groove, and when installed in the insertion groove, the O-ring is provided to protrude from the connection part, and the thickness (d2-d1) that the O-ring protrudes from the connection part is 0.1 mm to 0.9 mm.

2: The device of claim 1, wherein the installation space is provided to have the same size as the luminaire module, and the luminaire module is installed in the installation space in a fitting manner.

3: The device of claim 1, wherein the luminaire part comprises:
a reflector installed therein and configured to emit the light radiated from the luminaire module to the outside;
a fixing member provided on one surface of the reflector, provided with an installation space allowing the luminaire module to be installed therein, and configured to fix the luminaire module; and
a radiation hole provided between the reflector and a fixing member and provided to allow the light radiated from the luminaire module to be radiated in one direction.

4: The device of claim 3, wherein the luminaire module is installed in the installation space of the fixing member, but an LED installed in the luminaire module is installed to face the radiation hole, thereby allowing the light radiated by the LED to be radiated to the reflector through the radiation hole.

5: The device of claim 1, wherein the luminaire module generates UV rays to radiate to the outside, and
a wavelength of the UV rays is generated within a range of 380 to 390 nm.

6: The device of claim 1, wherein the housing is provided by being divided into a first housing and a second housing, and the first housing and the second housing are coupled to each other through a coupling means.

7: The device of claim 1, wherein the mounting means is configured by selecting any one of a clip, a magnet, and tongs.

8: The device of claim 1, wherein the fastening means is configured by selecting any one of a bolt, a screw, and a hinge structure.

9: The device of claim 1, wherein the thickness (d2-d1) that the O-ring protrudes from the connection part is 0.4 mm to 0.6 mm.

10: The device of claim 9, wherein the thickness (d2-d1) that the O-ring protrudes from the connection part is 0.5 mm.
